(19) United States
(12) Patent Application Publication
(10) Pub. No.: US 2004/0134008 A1
(43) Pub. Date: Jul. 15, 2004

(54) Combination tooth brush and tongue cleaner

(76) Inventor: Tuan Pham, Pearland, TX (US)

Correspondence Address: Tuan Pham, 2406 Wood Oak, Pearland, TX, 77584, US

(21) Appl. No.: 10/338,966

(22) Filed: Jan. 10, 2003

(51) Int. Cl. A46B 9/04; A61B 17/24

(52) U.S. Cl. 15/111; 606/161

(57) ABSTRACT

The combination tooth brush and tongue cleaner includes an elongated tubular handle portion having a first end which is closed and a second end opposite the first end which is open. A tooth brush portion extends linearly outwardly form the first end and has a plurality of upstanding bristles. A tongue cleaner takes form of an elongated, linear, flexible strip disposed in a cavity of the tubular handle portion. The strip has a proximal end attached to a movable retainer and a distal end connected to a finger puller which normally closed the open end of the handle portion whereby a finger puller is used to pull out the strip from the handle portion for tongue cleaning and push back the strip into the handle portion when tongue cleaning is complete.

### FIELD OF THE INVENTION

This invention relates broadly to improvements in the field of oral hygiene and, more particularly, pertains to an integral structure for permitting the cleaning of one's teeth and tongue.

### BACKGROUND OF THE INVENTION

The use of combined toothbrushes and tongue cleaners is known in the prior art. Toothbrushes heretofore utilized for the purpose of cleaning and brushing teeth and gums consist basically of familiar, expected and obvious configurations. In addition, tongue cleaners and scrapers are known and fabricated from a wide variety of materials, natural and synthetic, and are designed in various shapes and constructions. It the purpose of these toothbrush and tongue cleaners to eliminate bacteria which causes build up of plaque and halitosis or bad breath.

By way of example, U.S. Pat. No. 5,842,247 issued Dec. 1, 1998 to Decesare discloses a combination tooth brush and tongue brush. U.S. Pat. No. Des. 377,417 issued Jan. 21, 1997 to Gupta, U.S. Pat. No. Des. 283,952 issued May 27, 1986 to Berkowitz and U.S. Pat. No. Des. 265,270 issued Jul. 6, 1982 to McCarty all disclose combined tooth brush and tongue cleaners. Each of these designs shows a handle portion having a tooth brush at one end and an exposed tongue cleaner fixed at an opposite end.

While these prior art devices fulfill their respective objectives, the aforementioned patents do not show a combination tooth brush and tongue cleaner wherein the latter is of flexible construction and is selectively movable into and out of the handle portion of the tooth brush.

Accordingly, it should be understood that there exists a need for a differently styled combination tooth brush and tongue cleaner which can be utilized to remove plaque coating bacteria, reduce halitosis and lead to better overall mouth cleaning and oral hygiene.

### SUMMARY OF THE INVENTION

The present invention advantageously eliminates offensive odor in the mouth, functions to obtain and maintain adequate oral hygiene and abates some periodontal diseases.

It is a general object of the present invention to provide an integral tooth brush and tongue cleaner wherein a tongue cleaner is retracted and expanded from the handle portion of the tooth brush.

It is also an object of the present invention to provide a combined tooth brush and tongue cleaner which may be easily manufactured and marketed.

It is a further object of the present invention to provide a combined tooth brush and tongue cleaner which is of durable and reliable construction.

It is another object of the present invention to provide a home dental tool which will remove plaque-like patches from the teeth, gums and tongue.

It is an additional object of the present invention to freshen breath by eliminating the halitosis caused by plaque-like coating in the mouth.

It is yet another object of the present invention to provide a tongue cleaner more hygienically clean and safe by making the tongue cleaner storable in the handle portion of the combined tooth brush and tongue cleaner.

In one aspect of the invention, a combination tooth brush and tongue cleaner includes an elongated tubular handle portion having a first end which is closed and a second end opposite the first end which is open. A tooth brush portion extends linearly outwardly from the first end and has a plurality of upstanding bristles. A tongue cleaner takes the form of an elongated, linear, flexible strip disposed in a cavity of the tubular handle portion. The strip has a proximal end attached to a movable retainer, and a distal end connected to a finger puller which normally closes the open end of the handle portion whereby the finger puller is used to pull out the strip from the handle portion for tongue cleaning and push back the strip into the handle portion when tongue cleaning is complete.

Various other features, objects and advantages of the invention will be made apparent from the following description taken together with the drawings.

### BRIEF DESCRIPTION OF THE DRAWINGS

The drawings illustrate the best mode presently contemplated of carrying out the invention.

In the drawings:

FIG. 1 is an elevational view with parts broken away showing a combination tooth brush and tongue cleaner;

FIG. 2 is a view similar to FIG. 1 but showing a flexible tongue cleaner pulled from a storage position in the handle portion to an operative position outside the handle portion; and

FIG. 3 is an alternative embodiment of a combined tooth brush and tongue cleaner.

### DETAILED DESCRIPTION

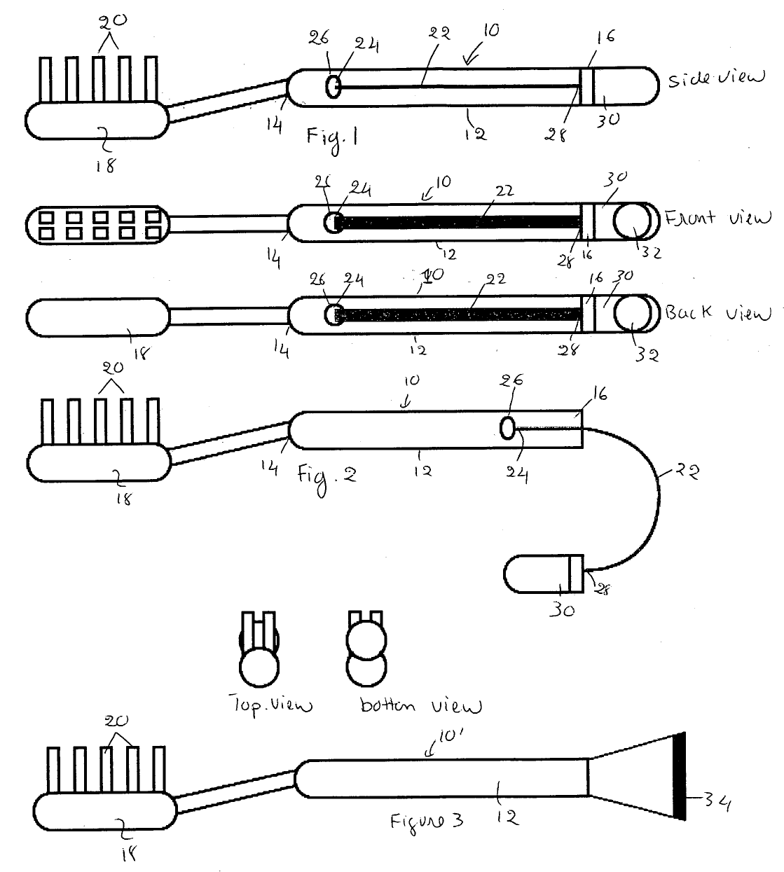

Referring now to the drawings, FIGS. 1 and 2 illustrate the new and improved combination tooth brush and tongue cleaner 10 embodying the principles and concepts of the present invention.

At the outset of this description, it should be understood that the mouth can become a breeding ground for bacteria to grow which allows the opportunity for the collection of germs, food debris, dead cells and microorganisms. Over time, some collected material can develop into plaque which makes a firm attachment to the teeth, tongue and gums. Elimination of plaque is important for overall oral hygiene, and the design of the present invention is intended to minimize such plaque and reduce the bacteria in one's mouth.

Combination cleaner 10 has a generally elongated, tubular handle portion 12 having a first end 14 which is closed, and a second end 16 which is open. Handle portion 12 is typically constructed of the heavy duty plastic material, as is known in the tooth brush art.

A tooth brush portion 18 extends linearly from the first end 14 of the handle portion 12 and includes a row of upstanding bristles 20. A tongue cleaner 22 takes the form of elongated, linear, flexible strip which is normally held in a storage position within the handle portion 12 when the tooth brush portion 18 is being used, and may be withdrawn from the handle portion 12 when it is desired to clean the tongue. The flexible strip 22 has a first or proximal end 24 attached to a movable retainer 26 which slides back and forth in the handle portion 12, and a second or distal end 28 attached to a finger puller 30 which normally closes the open end 16 of the handle portion 12. The finger puller 30 is provided with a throughhole 32 which can be engaged by one's finger to pull the tongue cleaner 22 out of the handle portion 12 and form a curved portion (FIG. 2) to be drawn across the tongue.

The preferred materials of the tongue cleaner 22 are propylene or polyethlene. Other cleaning materials include high density, polymer plastics which are FDA approved for consumer use. The material should be efficiently flexible so that the tongue cleaner 22 can assume the arcuate configuration as seen in FIG. 2.

Although not shown, the end 16 of the handle portion 12 may be provided with a floss dispenser.

In use, one brushes his or her teeth and gums employing the tooth brush portion 18 as is well known. When it is desired to clean the tongue, one places a finger in the finger puller 30 and pulls outwardly to expose the cleaner strip 22 which is subsequently bent into the arcuate configuration so that it can scrape the surface of the tongue. When cleaning the tongue, the cleaner 10 is typically held at the end 16 of the handle portion in one hand and at the finger puller 30 by the other hand. After tongue cleaning, the strip 22 is rinsed off and the finger puller 30 is used to push the flexible strip 22 back into the handle portion 12.

FIG. 3 shows an alternative embodiment of a combination tooth brush and tongue cleaner 10′ wherein the distal end of the handle portion 12 is provided with a fixed, trapezoidal-shaped tongue cleaner 34 having a roughened end surface 36 for cleaning the tongue.

With respect to the above description then, it is to be realized that the optimum conventional relationships for the parts of the invention to include variations in size, materials, shape, form, function and the manner of operation, assembly and use, are deemed readily apparent and obvious to one skilled in the art, and all equivalent relationships to those illustrated in the drawings and described in the specification are intended to be encompassed by the present invention.

While the invention has been described with reference to a preferred embodiment, those skilled in the art will appreciate that certain substitutions, alterations and omissions may be made without departing from the spirit thereof. Accordingly, the foregoing description is meant to be exemplary only, and should not be deemed limitative in the scope of the invention set forth in the following claim.

I claim:

1. A combination tooth brush and tongue cleaner comprising: an elongated tubular handle portion having a first end which is closed and a second end opposite the first end which is open;
a tooth brush portion extending linearly outwardly from the first end and having a plurality of upstanding bristles;
A generally elongated handle of the tooth brush is hollow for housing the tongue cleaner such that the tongue cleaner is normally hidden in the tooth brush;

2. The tooth brush and tongue cleaner combination of claim 1, wherein
a head portion of tooth brush includes a row of upstanding bristles for the very purpose of cleaning all the teeth.

3. The head portion of the tooth brush as in claim 2, further comprising of bristles that are constructed of plastic like material, as is known in the tooth brush art which also allows users to clean the tissue around the tooth if brush lightly.

4. The handle of the tooth brush as in claim 1, wherein is hollow to house the tongue cleaner in a hidden position. This handle said portion of toothbrush is typically constructed of the heavy duty plastic material, as is know in the tooth brush art.

5. The tongue cleaner of as in claim 1, wherein said takes the form of flat, elongated, liner, flexible strip which is normally held in a storage hidden position within the handle portion.

6. The tongue cleaner of as in claim 5, wherein the flexible strip may be withdrawn from handle portion when it is desired to clean the tongue. It mechanically comprising at one proximal end a movable retainer which slides back and forth in the toothbrush tubular handle. The other end-or distal end attached to a finger puller, which normally communicated in close position with the handle portion. The distal end of the finger puller has a through hole for engaged by one's finger to pull the tongue cleaner. This through hole can be used to hang the toothbrush on the wall as well-if desire by the user.

7. The tongue cleaner of as in claim 6, wherein the proximal end a movable retainer also served the purpose of helping secured the tongue cleaner in the tubular handle when not in used.

8. The tongue cleaner of as in claim 5, wherein should be constructed with preferred materials of propylene or polyethylene. Other materials include high density, polymer plastics which are approved for consumer use by FDA or light stainless steal. The constuction material also optional depending on future alternative embodiment design. Importantly, the material for construction should be efficiently flexible so that the tongue cleaner can assume the arcuate configuration.

9. In this unique combination of the toothbrush and a hidden tongue cleaner, wherein said that it served the innovated purpose of cleaning the teeth at the same time help to remind the users to clean the tongue as well.
